(19) United States
(12) Patent Application Publication
(10) Pub. No.: US 2023/0309837 A1
(43) Pub. Date: Oct. 5, 2023

(54) SYSTEMS AND METHODS FOR NONCONTACT ULTRASOUND IMAGING

(71) Applicants: Massachusetts Institute of Technology, Cambridge, MA (US); The General Hospital Corporation, Boston, MA (US)

(72) Inventors: Alan J. FENN, Wayland, MA (US); Robert W. HAUPT, Lexington, MA (US); Anthony SAMIR, Boston, MA (US); David J. BRIGADA, Nashua, NH (US); Rebecca LAHER, Cambridge, MA (US); Brian Boitnott, Cambridge, MA (US)

(21) Appl. No.: 18/127,884

(22) Filed: Mar. 29, 2023

Related U.S. Application Data
(60) Provisional application No. 63/324,833, filed on Mar. 29, 2022.

(51) Int. Cl. A61B 5/00

(52) U.S. Cl. CPC A61B 5/0093; A61B 5/0042

(57) ABSTRACT

Systems and methods for non-contact, non-invasive image construction of interior tissue are provided. Electromagnetic (EM) waves may be used to transmit through a high acoustic material or barrier, such as a bone, where the EM wave is then absorbed and converted to ultrasound (US) or audible band acoustic longitudinal waves or shear waves once past the high acoustic impedance barrier. The EM to acoustic converted waves are generated through thermoelastic mechanisms. This enables acoustic waves to propagate in the soft tissue on the opposing side of the barrier while minimizing reverberation and clutter. The US waves propagate within the tissue and may be measured using a detector, such as coherent lidar or optical band multipixel camera noninvasively outside the tissue. Furthermore, a phased array can be used to steer and shape the acoustic radiation pattern of the acoustic waves in the soft tissue beyond the bone or high acoustic impedance barrier.

### CROSS-REFERENCE TO RELATED APPLICATIONS

This application is based on, claims priority to, and incorporates herein by reference for all purposes, U.S. Provisional Application Ser. No. 63/324,833, filed Mar. 29, 2022.

### STATEMENT REGARDING FEDERALLY SPONSORED RESEARCH

This invention was made with government support under FA8702-15-D-0001 awarded by the U.S. Air Force. The government has certain rights in the invention.

### BACKGROUND

There is a need to expeditiously detect brain injury requiring treatment after head trauma such as intracranial hemorrhage (ICH). Such injuries often show no symptoms and remain undetected until emergency intervention is necessary. CT and MRI systems are highly sensitive and specific for ICH detection, but, may be impractical for field-forward patient examination. Medical ultrasound (US) is an ideal imaging modality that is portable, fast, inexpensive, safe, and produces images with excellent resolution. However, despite these advantages, noninvasive transcranial US is impractical due to the high acoustic impedance between the skull and brain. The skull can reflect US transmission into the brain and generates strong acoustic reverberation overwhelming signals of interest from the skull interior. If these limitations were overcome, US would revolutionize field forward neuroimaging for the warfighter and civilian populations.

Ultrasound is also viewed as having no known harmful biological effects, as long as exposures are kept within well-characterized safety limits. Although ultrasound use for body-scans of soft tissue has been widely successful, acquiring ultrasound images of the intracranial contents is extremely difficult using conventional ultrasound systems. These systems typically employ longitudinal or compressional waves that readily travel through body tissue, but do not easily traverse the calvarium. The large acoustic impedance that exists between the skull bone and fluid material surrounding the brain greatly in adults greatly suppresses subcranial acoustic signal transmission and return, reducing echo amplitude, and clarity when captured by a receiver at the skull outer surface.

Two fundamental forms of ultrasound signal interference are caused by this geometry that severely limit conventional ultrasound systems for brain imaging. First, the skull bone is relatively thin (only a few ultrasonic wavelengths) and, thus, ultrasonic waves tend to ring or reverberate over time as they bounce back and forth between the skull-brain and skull-exterior (air) interfaces-causing significant resonance-interference. Second, a variety of wave types are simultaneously induced by the ultrasonic source positioned at the skull exterior surface (longitudinal, shear, Rayleigh surface waves). These waves propagate away through very different travel paths (some travel along the skull surface, others travel inside the skull as guided waves, others transmit across the skull). When these signals return to the receiver, they mix and interfere with each other introducing numerous artefacts that prevent echolocation and appropriate assignment of echo amplitude to specific regions. The result is a set of challenges collectively termed “inline plane-interference.” These above-mentioned forms of interference (resonance and inline-plane) together greatly diminish the signal-to-noise ratio (SNR) of transcranial ultrasound overwhelming the signal of interest from ICH.

Thus, there remains a need for a non-invasive method for imaging a subject, such as through a skull, that provides the same or similar advantages to US, but with the ability to achieve meaningful, high-resolution images.

### SUMMARY OF THE DISCLOSURE

The present disclosure addresses the aforementioned drawbacks by providing systems and methods for non-contact imaging that utilizes wave conversion. In some non-limiting configurations, electromagnetic (EM) waves are used to transmit past a barrier, such as a skull of a subject, where the RF is absorbed and converted to US waves once past the barrier. This approach enables acoustic energy to be well-coupled to tissue on the opposing side of the barrier, such as brain tissue within a skull, while controlling against reverberation and clutter. The US waves propagate within the tissue and can be measured using coherent lidar, for example. The lidar wavelength may be selected to enable transmission through a portion of the barrier, such as through a calvarium into the cranial cavity. The US wave may modulate the optical wave, which can then be received noninvasively outside the skull upon return. In a non-limiting example, the skull layer is effectively eliminated by use of the methods in accordance with the present disclosure, permitting sonographic imaging of the brain. In some configurations, the system may be portable for use in field-forward settings as a means to detect and image ICH.

The systems and methods may facilitate measuring subtle acoustic contrasts from tumors and other diseases of brain tissue. The systems and methods may also provide for detecting treatable head injuries in civilian and military applications at locations away from the hospital setting. A noninvasive approach to US for brain imaging and diagnostics may provide medical staff a tool to detect dangerous hematomas in the field. In some configurations, a system may include low cost, low swap, and may be portable. In some configurations, tumors and other disease states may be monitored.

In one aspect, a method is provided for generating at least one of an image, or a tissue map of a subject, and/or providing diagnostic information characterizing interior tissue disease with the method comprising: transmitting EM waves to a subject without patient contact, external to the human body. The method includes generating thermoelastic acoustic propagating waves inside the subject using the EM waves as the source; detecting and measuring the acoustic propagating waves using an optical device or a contact transducer system to sense, temporally measure, and spatially map acoustic/mechanical vibrational waves. The method also includes construction of at least one image, tissue characterization or report of the subject based on the sensed and measured acoustic propagating waves.

In one aspect, a method is provided for generating an image or a map of a subject. The method includes delivering a first electromagnetic radiation to a first material in the subject and converting the first electromagnetic radiation to an acoustic radiation force to transmit within a second material in the subject. The method also includes detecting transmission of the acoustic radiation force within the second material in the subject to acquire data and generating an image or a map of the subject from the data.

In one aspect, a system is provided for generating at least one of an image or a map of a subject. The system includes a first electromagnetic radiation transmitter for delivering a first electromagnetic radiation to a first material in the subject. The first electromagnetic radiation is configured to convert to an acoustic radiation force to transmit within a second material in the subject. The system also includes a detector for detecting transmission of the acoustic radiation force within the second material in the subject to acquire data. The system also includes a computer system configured to generate an image or a map of the subject from the data.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 is a block diagram of a non-limiting example system for non-contact, non-invasive imaging.

FIG. 2 is a flowchart of non-limiting example steps for a method for non-contact, non-invasive imaging.

FIG. 3A is a diagram and a graph of pulsed RF with resulting elastic deformation in a subject.

FIG. 3B is a graph of non-limiting example RF decay with penetration depth in tissue.

FIG. 4 depicts graphs of electrical properties for non-limiting example brain gray matter, white matter, and zerdine.

FIG. 5 is a diagram of a non-limiting example transmitter with an imaging phantom and contact transducer.

FIG. 6 is a diagram of non-limiting examples of contact transducers.

FIG. 7 is a graph of ultrasound signals travelling through the phantom shown in FIG. 5.

FIG. 8A is a set of graphs showing measured RF to ultrasound signals.

FIG. 8B show a non-limiting resultant broadband acoustic wave and ultrasound spectrum measured by a contact transducer placed at the far end of a phantom as shown in FIG. 5 using an RF power of 2000 W and 100 W.

FIG. 8C shows a non-limiting example of modeled tissue heating using an RF power of 2000 W and 100 W.

FIG. 9A is an image of a non-limiting example RF antenna along with an associated radiation pattern.

FIG. 9B are perspective views of non-limiting example water-filled circular waveguide applicators.

FIG. 9C is a perspective view of an array of non-limiting example circular waveguides.

FIG. 10 is a graph of a non-limiting example RF measured reflection coefficient vs. frequency for a non-limiting RF antenna shown in FIG. 9A.

FIG. 11A is a non-limiting example graph of the optical penetration in tissue.

FIG. 11B is a non-limiting example graph of the optical penetration in bone.

FIG. 12 is non-limiting example time snapshots of an acoustic waveform from four RF sources and thirty RF sources.

FIG. 13 is non-limiting example graphs of acoustic max power for 4 and 30 RF source antenna distributions.

FIG. 14 is non-limiting example ultrasonic wave propagation time snapshots from a 2D simulation are shown for frontal and side excitations of a subject's skull.

FIG. 15A is a graph illustrating time-series signals using wavelet analysis.

FIG. 15B is a non-limiting example of an ultrasound image using synthetic aperture ultrasonic image construction.

### DETAILED DESCRIPTION

Systems and methods for non-contact, non-invasive imaging are provided. Electromagnetic (EM) waves may be used to transmit past a barrier, such as a skull of a subject, where the RF is absorbed and converted to ultrasound (US) waves or shear waves once past the barrier. This approach enables acoustic energy to be well-coupled to tissue on the opposing side of the barrier while minimizing reverberation and clutter. The US waves propagate within the tissue and may be measured using an optical detector, such as coherent lidar. The lidar wavelength may be selected to enable transmission through a portion of the barrier. The US wave may modulate the optical wave, which is then received noninvasively outside the tissue upon return. In some configurations, the system may be portable for use in field-forward settings as a means to detect and image ICH.

In some configurations, RF waves are used to transmit past the skull, absorb, and convert to US waves once inside the brain. This enables acoustic energy to be well-coupled to brain tissue, while, minimizing skull reverberation and clutter. The US waves propagate within brain tissue and are then measured using coherent lidar. The lidar wavelength may be selected to enable transmission through the calvarium into the cranial cavity. The US wave modulates the optical wave, which is then received noninvasively outside the skull upon return. The skull layer is effectively eliminated, permitting sonographic imaging of the brain. A portable system may be used in field-forward settings as a means to detect and image ICH. Subtle acoustic contrasts may be measured from tumors and other diseases of brain tissue.

The systems and methods may facilitate measuring subtle acoustic contrasts from tumors and other diseases of brain tissue. The systems and methods may also provide for detecting treatable head injuries in civilian and military applications at locations away from the hospital setting, such as TBI, ICH, and internal bleeding. A noninvasive approach to US for brain imaging and diagnostics may provide medical staff a tool to detect dangerous hematomas in the field. In some configurations, a system may include low cost, low swap, and may be portable. In some configurations, tumors and other disease states may be monitored that may be unobservable with conventional ultrasound due to the attenuation typically suffered by conventional ultrasound when passing through a barrier, such as a bone or skull. Elastography may also be used to determine tissue mechanical properties for tumor detection, progression, and classification.

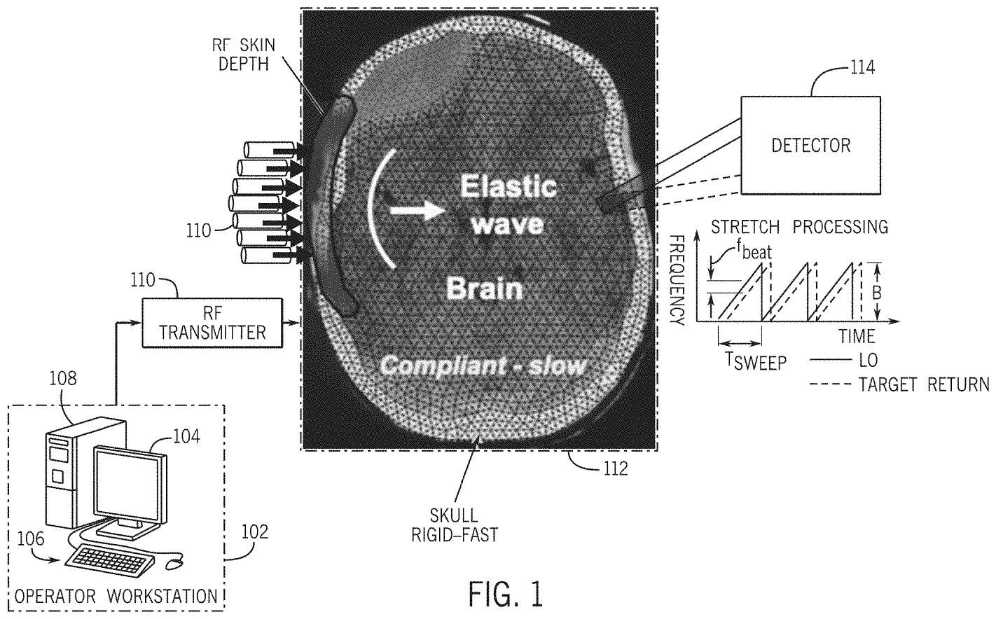

Referring to FIG. 1, a non-limiting example system for noncontact RF to ultrasound imaging is shown. A control system 102, such as a workstation, server 108, computer system, imaging system server, or other appropriate control system may include an interface 106, such as a keyboard, mouse, or other interface, and may include a display 104. Control system 102 is configured to direct the transmitter 110 to produce RF and direct the RF to a subject 112. In a non-limiting example, the subject 112 includes a skull and the RF is directed to the skull to produce ultrasound waves within the skull. The RF directed to the subject 112 produces ultrasound waves that are detected by detectors 114.

Ultrasound imaging or elastography may be performed in accordance with the present disclosure. Ultrasound imaging may include echo-pulse, tomographic, or any other ultrasound imaging form. Elastography may include shear wave conversion, hematoma detection, tumor detection or grading, disease state determination, and the like.

A transmitter may include a carrier frequency of 20 kHz-10 GHz. For example, audible frequencies can range from 20 Hz to 20 kHz, ultrasonic frequencies can rage form 20 kHz to 10 MHz, and the carrier can include these ranges and/or others. In a non-limiting example, the transmitter is configured to emit 2 GHz waves. In another non-limiting example, the transmitter is configured to emit 1.6 GHz waves. In some configurations, an array or a plurality of transmitters may be used. The transmitters or antenna may be spaced apart to deliver a desired wave configuration to the subject, such as a plane wave. In a non-limiting example, spacing may be 0.1-0.8 of the RF wavelength.

In some configurations, transmitters or applicators may be used to generate 1-5 mm spot size beams outside a skull that transmit across the skull and then convert to ultrasound once inside the brain. The ultrasound waves then travel and interact in tissue like standard ultrasound.

In some configurations, an optical detector such as a coherent laser vibrometer, or light detection and ranging (LIDAR) detector, may be used to measure the ultrasound waves just inside the skull at a prescribed datum. The optical carrier wavelength of the laser may be selected as a means to penetrate through the skull. In a non-limiting example, the selected wavelength may be 700-1064 nm, or may be selected to be in the range of 700-800 nm. The power of the optical wavelength may be selected to be skin safe, but sufficient to overcome the significant loss of two-way transmit through the skull. In some configurations, this may be accomplished through time and multipixel averaging. In some configurations, the optical detector or laser may include a swept sine or ramp to provide for range binning of the detected waves, which provides for determining a depth of a feature in the subject.

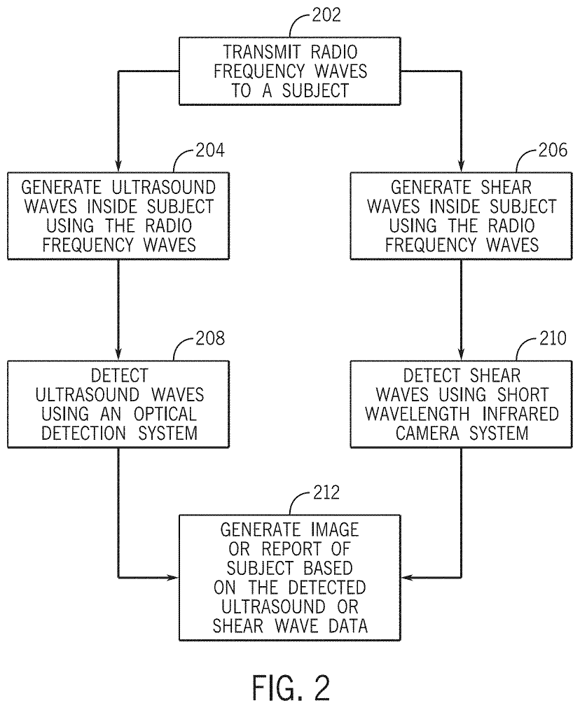

Referring to FIG. 2, is a flowchart of non-limiting example steps for a method for non-contact, non-invasive imaging. RF may be transmitted to a subject at step 202. In a non-limiting example, the subject includes a skull, and the RF is transmitted into the skull of the subject. Ultrasound waves may be generated inside the subject at step 204 using the RF waves. These ultrasound waves may be detected using an optical detection system at step 208. In a non-limiting example, the optical detection system is a LIDAR detector. Shear waves may also be generated inside the subject using the RF waves at step 206. These shear waves may be detected using a optical detection system at step 210. In a non-limiting example, the shear wave optical detection system is a short wavelength infrared camera (SWIR) system. An image or report of the subject may be generated at step 212 based on the detected ultrasound or shear wave data. In non-limiting examples, the image may be an ultrasound image of the subject, the report may be an elastogram or depiction of the stiffness of the subject based on the shear wave data, the report may be a depiction of wave speed data for the ultrasound waves or shear waves, the report may include a stiffness report of the subject, and the like.

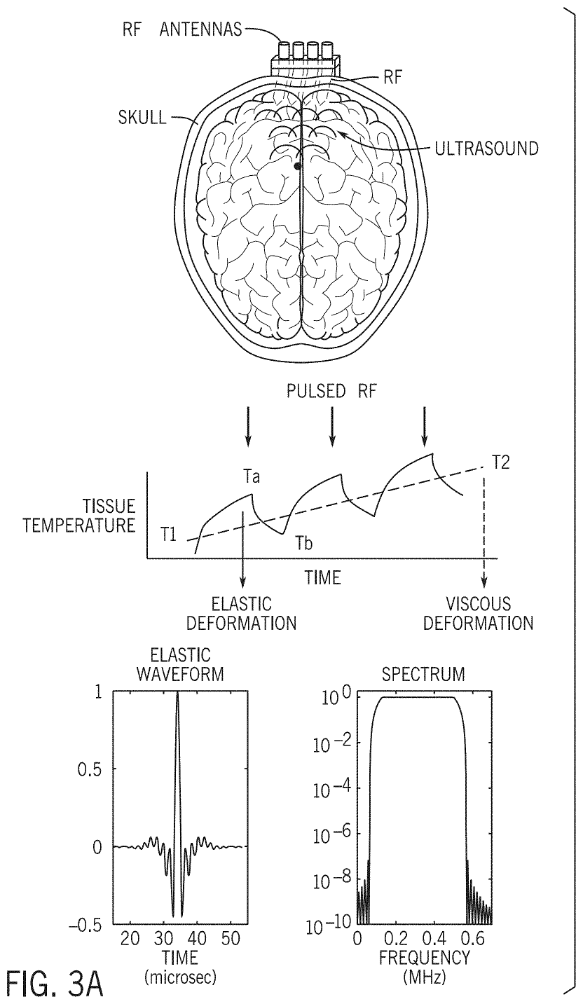

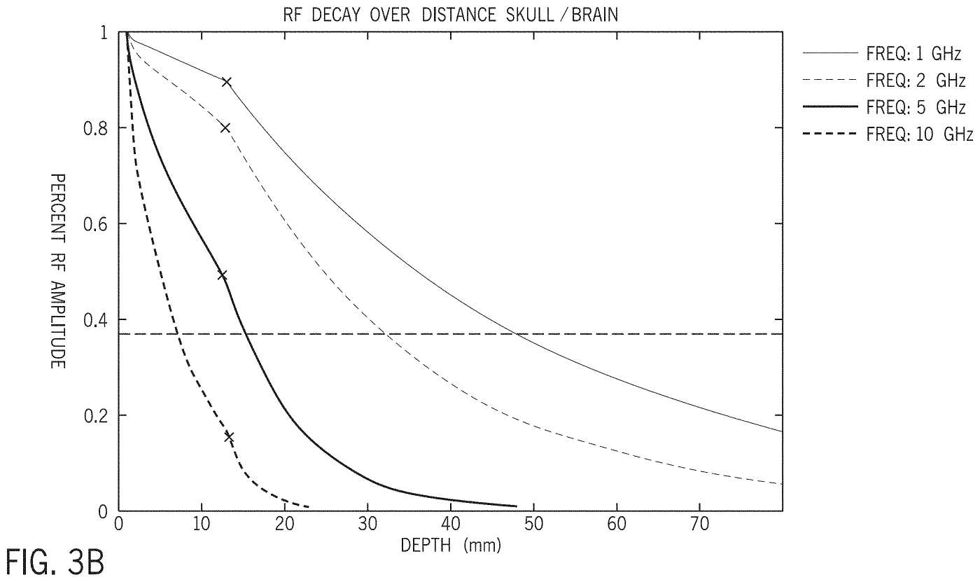

Referring to FIG. 3A, a graph of pulsed RF with resulting elastic deformation in a subject is shown. In some configurations, converting a transmitted RF to US within a subject may include pulsing the delivered RF. This may result in an elastic deformation of the tissue of the subject. Non-limiting example pulse widths include widths selected to be 100 nanoseconds-10 microseconds. Graphs of a non-limiting example resultant acoustic elastic waveform and spectrum are also shown. Converting RF to US may include determining a pressure resulting from the transmitted RF.

The RF to pressure conversion may be determined by:

P₀=ΓμαF, where Γ=βν_{S}²/Cₚ (1)

The pressure wave may be determined by:

$${∇}^{2} p ( r , t ) - \frac{1}{{v}_{s}^{2}} \frac{{\partial }^{2} p ( r , t )}{\partial {t}^{2}} = \frac{β}{{C}_{p}} [ {μ}_{a} + {Δμ}_{a} ( r ) ] \frac{\partial F ( r , t )}{\partial t} . \qquad ( 2 )$$

Where p represents pressure, Γ represents a Gruneisen parameter of tissue, μₐ represents an RF absorption coefficient, F represents local RF fluence, β represents a volume expansion coefficient, vs represents an elastic wave speed, and Cₚ represents specific heat of the tissue.

Referring to FIG. 3B, a non-limiting example graph of RF decay with penetration depth in tissue is shown. Specific Absorption Rate (SAR) is the rate at which RF energy is absorbed by human tissue. The SAR is dependent on the RF exposure conditions and characteristics of the tissue itself. A specific absorption rate may be determined by:

$$SAR = \frac{σ ( r ) {\vert E ( r ) \vert}^{2}}{ρ ( r )} = c \frac{dT}{dt} \qquad ( 3 )$$

Where σ represents sample electrical conductivity, E represents RMS electric field, ρ represents sample density, c represents specific heat of tissue, dT represents change in temperature, dt represents change in time.

A safe SAR limit for a whole-body average may be a maximum permissible exposure of 0.4 W/kg, and a local SAR (per kg of tissue) limit may be a maximum permissible exposure of 8.0 W/kg. Other safety considerations include tissue heating, where cell temperature may increase due to RF absorption. The safety threshold for temperature increase may be <42° C. For genotoxicity, safety considerations may include consideration of micronucleus formation, DNA strand breaks, and chromosome damage.

Ultrasound safe limits may be determined by the Mechanical Index (MI), which is the maximum amplitude of the pressure pulse in the body, which may be given by:

$$MI = \frac{{P}_{r}}{\sqrt{{f}_{c}}}$$

Where Pᵣ represents peak rarefaction pressure of an ultrasound wave, ƒ_{c} represents the ultrasound wave center frequency.

Ultrasound safe limits may include consideration of mechanical stress from acoustic radiation force, such as with a MI threshold of <1.9, above which tissue damage can occur, or cellular tear may take place. Thermal effects may also be considered, such as tissue temperature increase by mechanical friction. As noted above regarding SAR, a safety threshold may be determined as <42° C. Cavitation may also be considered, where vapor-filled bubbles can cause tissue damage. Auditory and vestibular effects may also be considered, such as taking into account anticipated perception of audible clicks detected in the cochlea and vertigo, or other cognitive impacts.

Optical safe parameters may also be considered. Tissue heating may be considered when considering a laser beam footprint on skin, which can burn with excessive optical power. As with the above considerations, a safe temperature limit for optical power considerations may be <42° C. Skin exposure may also be considered based on a risk of skin cancer development with prolonged exposure times. Eye exposure may create retina photoreceptor cell damage or cell death. A laser aperture may be adjusted in order to avoid unnecessary eye exposure, such as a 3 mm beam diameter or less permitted to enter a pupil. A safe optical intensity may be determined by:

$$I = \frac{{Pd}^{2}}{{f}^{2} {λ}^{2}}$$

Where P represents power; d represents spot size, f represents focal length, and λ represents optical wavelength.

TABLE 1
Non-limiting example tissue characteristics.

| | | Conduc- | | Specific Heat |
|---|---|---|---|---|
| | | tivity | Density | Capacity c |
| | Tissue Name | (S/m) | (kg m⁻³) | (J/kg ° C.) |
| Brain | Brain Gray Matter | 1.5111 | 1030 | 3640 |
| | Brain White Matter | 1.0014 | 1030 | 3640 |
| | Cerebrospinal Fluid | 3.0741 | 999.5 | 4186 |
| Blood | Blood | 2.1861 | 1050 | 3610-3890 |
| | Blood Vessel | 1.1708 | 1102 | 3306 |
| Skull | Cortical Bone | 0.31007 | Skull: 1912 | Skull: 1440 |
| | Bone Marrow | 0.07615 | | |
| | Cancellous Bone | 0.6522 | | |
| Eye | Lens | 1.2485 | 1050 | 3000 |
| | Cornea | 1.9837 | 1050 | 4178 |

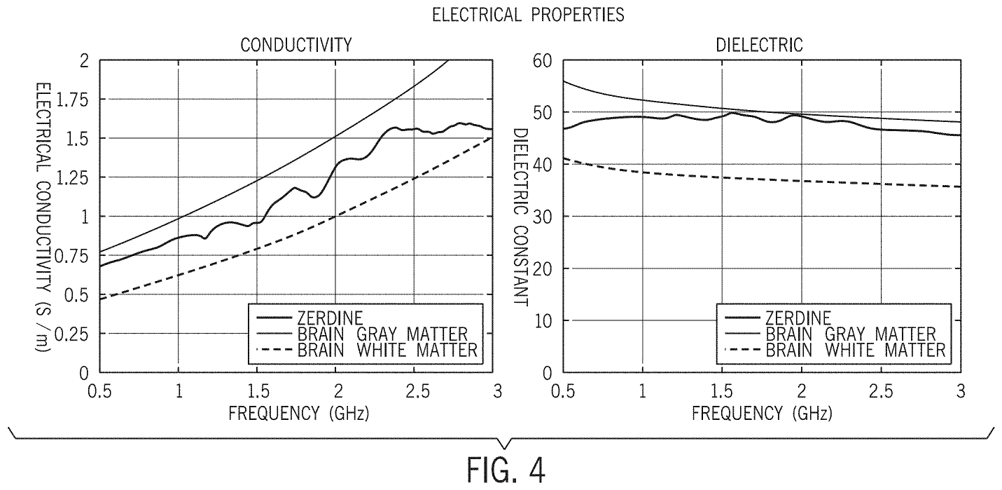

Referring to FIG. 4, graphs of electrical properties for non-limiting example brain gray matter, white matter, and zerdine are shown. Mechanical properties may also be determined for a subject in accordance with systems and methods of the present disclosure. Non-limiting example mechanical parameters for select materials are shown in Table 2.

TABLE 2
non-limiting example mechanical properties
Mechanical Properties

| | compressibility | | Specific | RF attenuation |
|---|---|---|---|---|
| | β | velocity | heat Cp | 2 GHz |
| material | (C⁻¹) | (m/s) | (J/kg/C.) | (m⁻¹) |
| Skull | 2.75 × 10⁻⁸ | 2964 | 1313 | 16.99 |
| Quartz | 5.5 × 10⁻⁷ | 5900 | 670 | 0.94 |
| Brain | 12.3 × 10⁻⁵ | 1540 | 3700 | 39.9 |
| Zerdine | 6.9 × 10⁻⁵ | 1506 | 4178 | 34.8 |

Non-limiting example brain tissue acoustic reflection imaging.

In a non-limiting example, RF energy may be directed to focus longitudinal ultrasound waves that propagate inside a brain cavity. The RF to US system may be operated without physical contact on the external side of the skull. Coherent-Lidar may be used to measure the converted acoustic/ultrasound wave. The lidar may use an optical wavelength of 810-1064 nm carrier which can propagate through the skull, such as at a depth of 0.5-2 cm, and measures the acoustic wave interference with brain tissues and anomalies. The lidar may also be operated without physical contact on the external side of the skull. In some configurations, the coherent lidar may use a linear chirp waveform which can range resolve the acoustic return. The range bins may be designed to provide an acoustic datum which then yields pertinent information that can be used to construct the ultrasound image of the brain tissue and cavity.

Non-limiting example RF to US shear wave elastography

In a non-limiting example, RF energy may be directed to focus longitudinal US wave, such as a 100 kHz wave, for performing elastography. A longitudinal wave creates force that launches low frequency shear waves. The shear waves may have a frequency of 10 -200 Hz. Short Wavelength Infrared (SWIR) Camera light may be used to detect the propagating shear waves. In a non-limiting example, the SWIR light may be used to penetrate a skull and measure a shear wave spatial and temporal speckle pattern during propagation. The SWIR camera may be selected to use 810-1064 nm wavelength Can penetrate skull and spatially images slow shear wave Speckle field as a function of time. SWIR Camera frame rate is set at 1 kHz and records speckle image of propagating shear wave. 2DFFT of time varying shear speckle field yields shear wave dispersion and characterizes hematoma and surrounding brain tissue

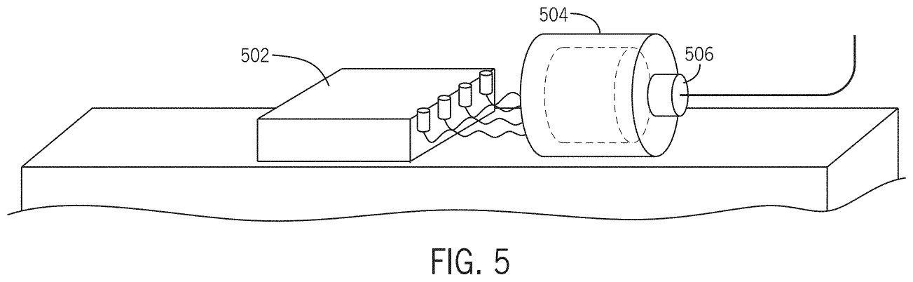

Referring to FIG. 5, a non-limiting example transmitter 502 with an imaging phantom 504 is shown. The transmitter 502 is configured to transmit RF waves into the phantom 504 to generate ultrasound waves inside the phantom 504. A detector 506 is configured to detect the ultrasound waves inside the phantom 504.

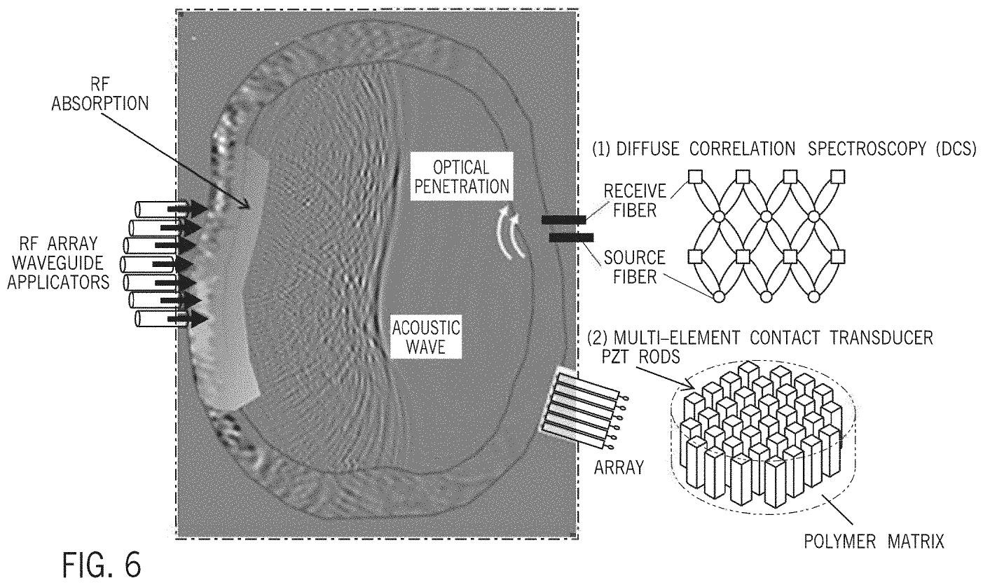

In a non-limiting example, the detector 506 is contact transducer receive array positioned on the external surface of the phantom or subject's scalp. Further, the contact transducer may measure the acoustic propagating waves on the exterior surface. In one example the contract transducer may include a wearable device or a flexible ultrasound receiver surface device. An example multi-element contact transducer is shown in FIG. 6.

In another non-limiting example, the detector 506 may be a diffuse correlation spectroscopy (DCS) system as shown in FIG. 6 including transmit and receive optical fiber that are in direct contact with the exterior of the phantom or scalp of a subject. In this example, the light from the transmit optical fiber can penetrate the skull. This light is then modulated by the ultrasound vibration induced via the RF signal, which is transmitted back to the receive optical fiber outside the skull.

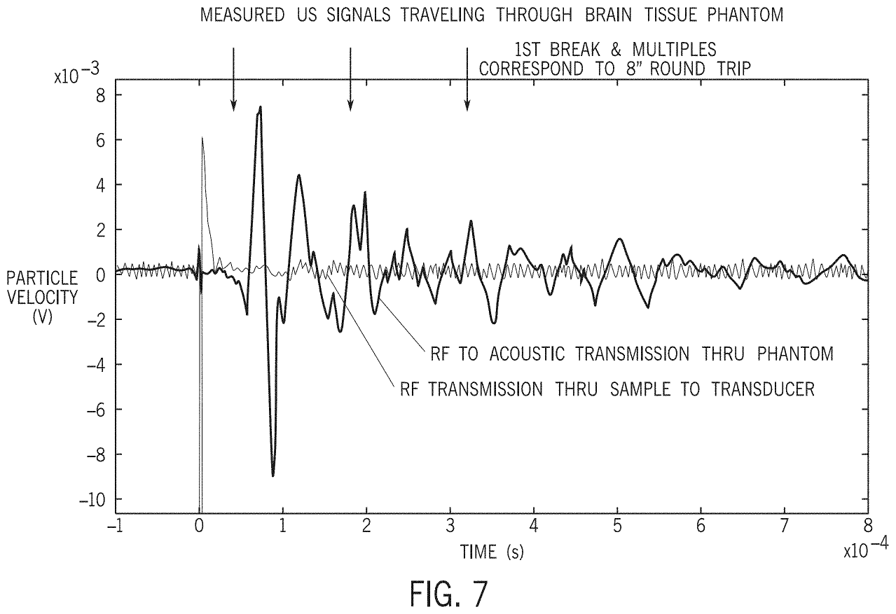

Referring to FIG. 7, a graph of ultrasound signals travelling through the phantom shown in FIG. 5 is shown. The graph shows that US signals travelling through the phantom include multiple reflections or “ringing.” Each reflection or harmonic may be processed to improve image quality, elastography data processing, and the like.

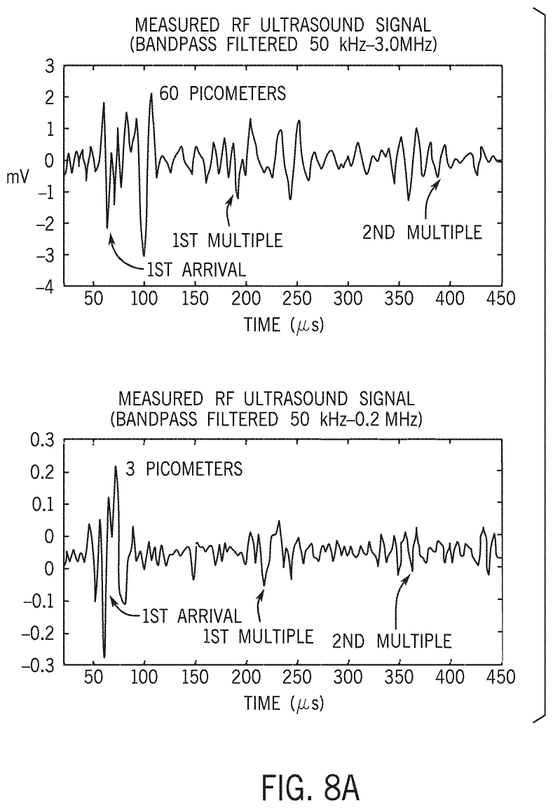

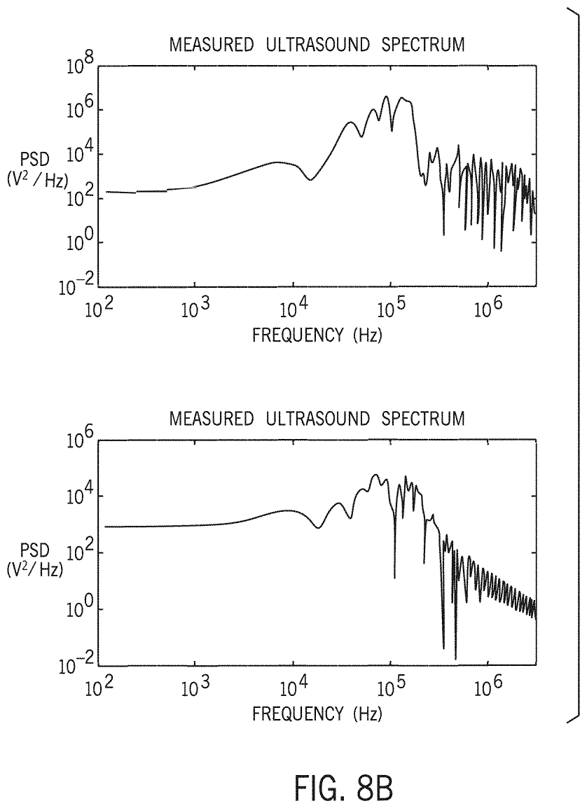

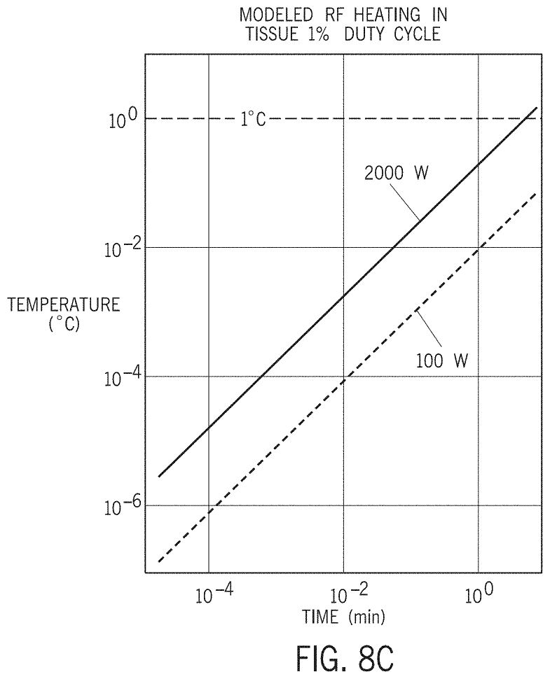

In a non-limiting example, FIGS. 8A-8C show the resultant broadband acoustic wave measured in a phantom by the contact transducer. FIG. 8A shows the measured RF to ultrasound signal acoustic time series using an RF power of 2000 W (top) and 100 W (bottom). US amplitudes below 100 picometers of displacement are well below concern for medical US tissue damage.

FIG. 8B shows the measured ultrasound spectrum by the contact transducer corresponding to the RF power of 2000 W (top) and 100 W (bottom). The resultant acoustic wave yields frequencies ranging from 30 kHz-300 kHz which can be used as a component to form an anatomical brain tissue image with a spatial resolution of 1 cm. In a non-limiting example, this spatial resolution may be useful for detecting, mapping, and characterizing hematomas or intracerebral hemorrhage in the cranial cavity. In a non-limiting example, the measurement of 1 MHz improves the spatial resolution to 1 mm.

FIG. 8C shows a non-limiting example of the modeled RF heating in tissue with a 1% duty cycle of the 2000 W and 100 W RF power. In this example, brain tissue heating from the 100 W RF sources is predicted to be below 1 using a 1% duty cycle for several minutes of excitation.

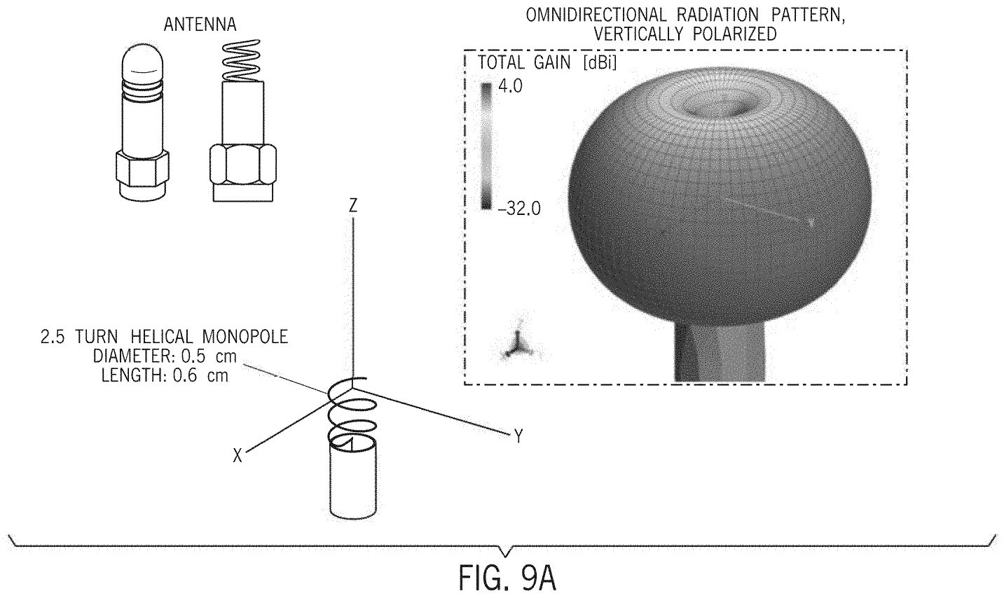

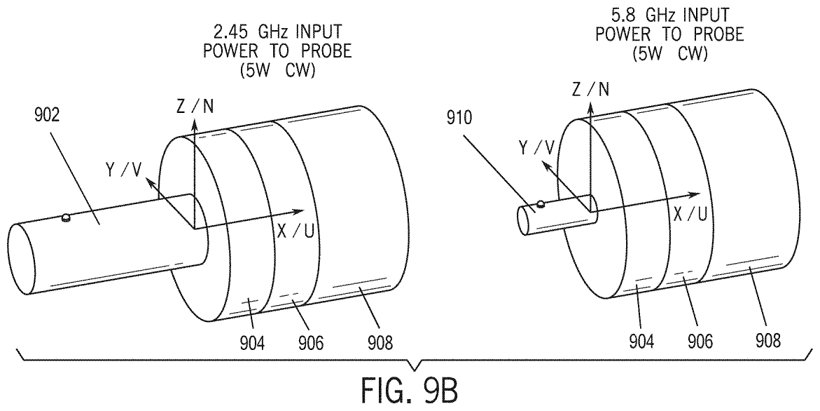

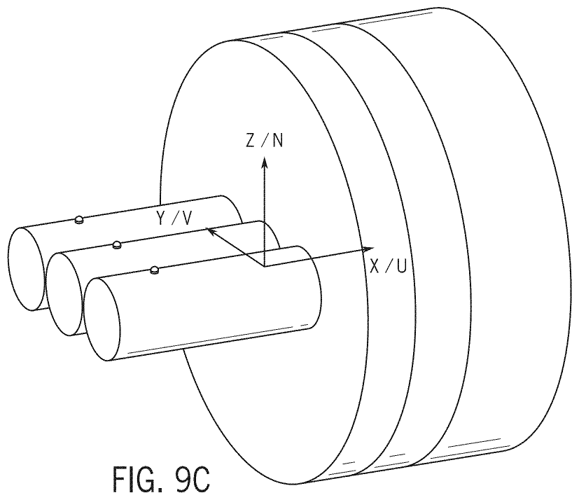

Referring to FIG. 9A, a non-limiting example RF antenna is shown along with an associated radiation pattern. The example RF antenna is shown as a helical monopole antenna with multiple turns. In a non-limiting example, the helical monopole antenna may be a 2.5 turn antenna with a diameter of 0.5 cm and a length of 0.6 cm. A circular coaxial waveguide may be used to couple the RF antenna to the power source and amplifier. In a non-limiting example, the circular coaxial waveguide may be 0.5 cm in diameter and 0.8 cm in length. The omnidirectional radiation pattern is shown vertically polarized for the non-limiting example helical monopole RF antenna. In some configurations, a system may use a phased array of a number of monopole antennas, such as 4 monopole antennas, that generate 1-5 mm spot size beams on the skull/brain region.

Referring to FIG. 9B, non-limiting example water-filled circular waveguide applicators are shown, in which the dominant field mode in the waveguide is the transverse electric (TE) fundamental mode, referred to as the TE11 mode. The circular metallic waveguide may have an open radiating end pointed at the body tissue and the opposite end may be closed presenting a short circuit to the field. At a given microwave frequency, and taking into account the dielectric constant ϵᵣ of water, a wire probe may couple power into the waveguide when the probe length is equal to approximately one-quarter wavelength λ/4 where λ=λ₀/√ϵᵣ and where λ₀ is the free space wavelength. For maximum field propagating toward the open end (aperture), the wire probe may be located a distance to the closed end of approximately one-quarter of the guide wavelength λ_{g}. The guide wavelength λ_{g} refers to the wavelength for the field propagating along the axis of the circular waveguide. From database parametric information at 2.4 GHz the average dielectric constant of white and gray matter brain tissue is 45 and the electrical conductivity is 1.5 Siemens/meter. Similarly, at 5.8 GHz again using average values, the dielectric constant is 40 and the electrical conductivity is 4.0 Siemens/meter.

In a non-limiting example, a FEKO multilevel-fast-multipole-method (MLFMM) surface equivalence principle simulation model at 2.45 GHz was used in which a single water-filled circular waveguide 902 with inner diameter 0.9525 cm [0.375 inches] was positioned adjacent to a deionized water bolus 904 that is next to the skull (bone) 906 followed by a volume of brain tissue 908 represented by the average dielectric parameters of gray and white matter. The water bolus thickness was 0.635 cm [0.25 inches], the skull (bone) thickness was 0.7 cm [0.275 inches], the brain thickness was 1.27 cm [0.5 inches]. The diameter was 2.54 cm [1 inch] each for the simulated water bolus, skull, and brain. The dielectric constant of the deionized water was assumed to be 80 and was lossless, such that the conductivity was zero. The dielectric constant of bone was assumed to be 11.7 with conductivity 0.41 Siemens/meter. The simulated transmit power was 5 Watts at the single frequency 2.45 GHz continuous wave (CW) in the Industrial Scientific Industrial (ISM) band. The wavelength in the dielectrically loaded circular waveguide was 1.37 cm [0.54 inches], and the calculated guide wavelength was 2.54 cm [1.0 inch]. The specific absorption rate (SAR) was proportional to the electrical conductivity times the electric field magnitude divided by the tissue density and was used to define the effective heating zone. The SAR was computed at a depth of 0.3175 cm [0.125 inches] in the brain.

In another non-limiting example, a FEKO simulation model was used in which the circular waveguide 910 had inner diameter 0.4 cm [0.158 inches] for operation in the ISM band at 5.8 GHz. The wavelength in the dielectrically loaded circular waveguide was 0.58 cm [0.23 inches], and the guide wavelength was 1.07 cm [0.42 inch]. For the 2.45 GHz simulation model of the single circular waveguide and phantom, the simulated SAR at 0.3175 cm depth was determined. For the 5.8 GHz simulation model, the simulated SAR at 0.3175 cm depth was determined. The simulated heated zone at 5.8 GHz was significantly smaller than the heated zone for the 2.45 GHz applicator.

Referring to FIG. 9C, an array of non-limiting example circular waveguides is shown. In some configurations, a method for moving the SAR beam peak position may be to transmit with only one element at a time. In some configurations, a method for moving or shaping the SAR beam peak position between two array waveguide elements may be to transmit from two elements with equal phase or variable microwave phase shift between the array elements. In some configurations, a method may be to transmit from the entire array with variable microwave phase shift between the elements to focus the peak heated zone within a subject, such as in brain tissue.

In a non-limiting example, a three-element water-filled circular waveguide array was simulated at 2.45 GHz. The simulated specific absorption rate (SAR) for a 3-element array of water-filled circular waveguide applicator operating at 2.45 GHz with focused beam steering produced by transmitting from two elements was determined. The center element and one element on the left were transmitting with equal power and equal phase. The microwave beamsteered peak SAR occurred at a position between the two transmitting elements.

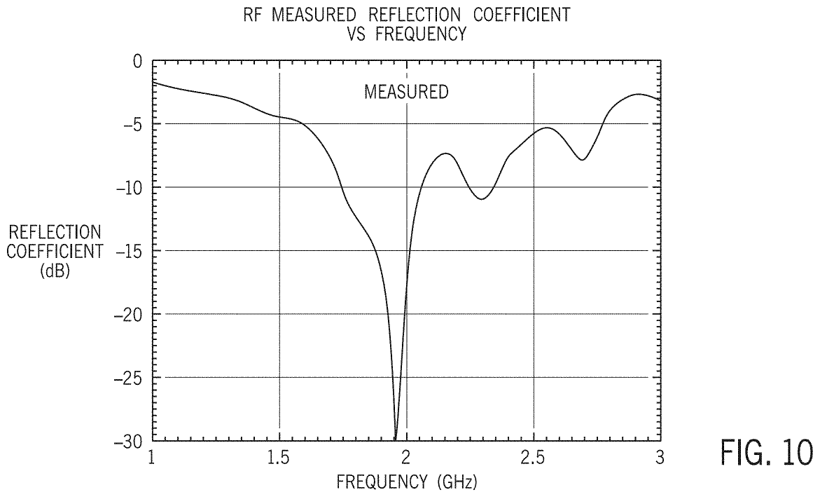

Referring to FIG. 10, a graph of a non-limiting example RF measured reflection coefficient vs. frequency is shown for a non-limiting RF antenna shown in FIG. 9A. In this example, the design is optimized for 2 GHz RF transmission. For example, the length of a bullet shaped element that contains a simple electrically conductive helical wire that can be driven at several GHz to produce an RF carrier signal can be adjusted to generate a 2 GHz RF signal.

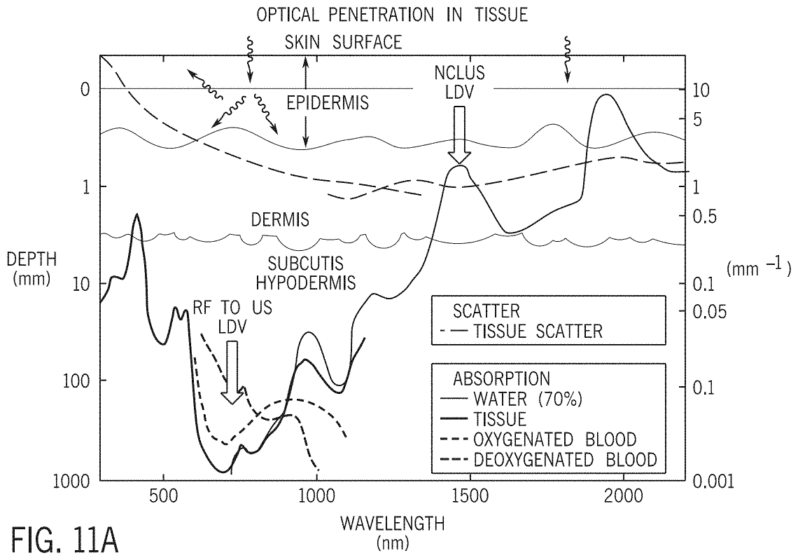

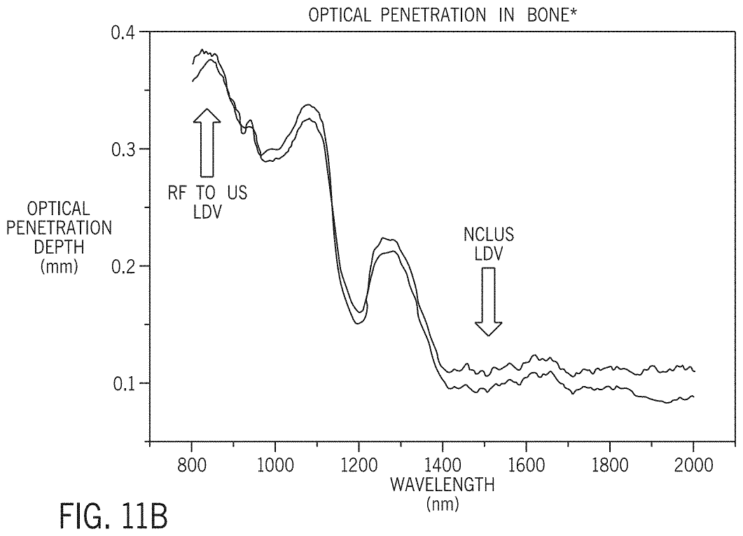

Referring to FIG. 11A, and 11B, non-limiting example graphs of the optical penetration in tissue and bone, respectively, are shown.

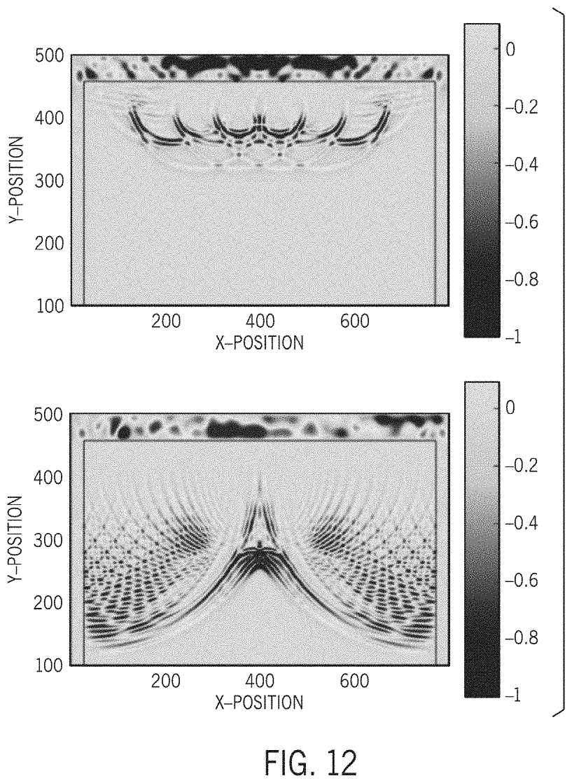

Referring to FIG. 12, non-limiting example time snapshots of an acoustic waveform from four sources and thirty sources are shown.

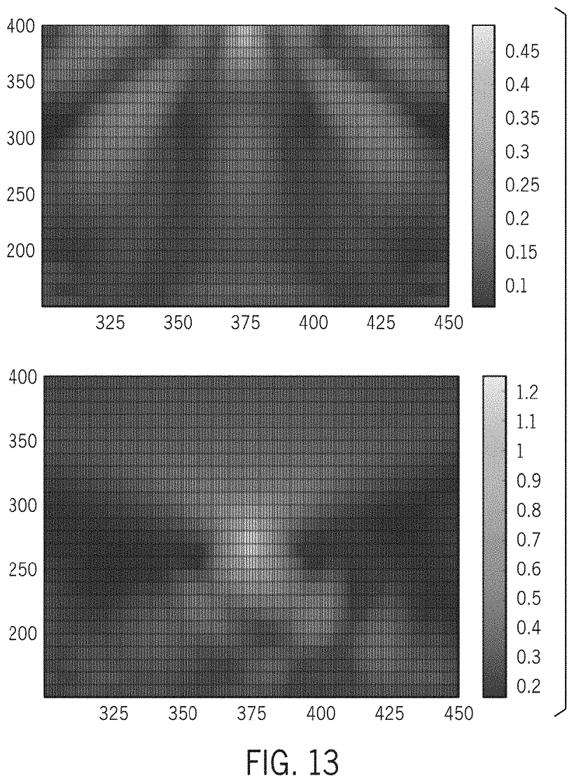

Referring to FIG. 13, non-limiting example graphs of acoustic max power for 4 and 30 source antenna distributions are shown.

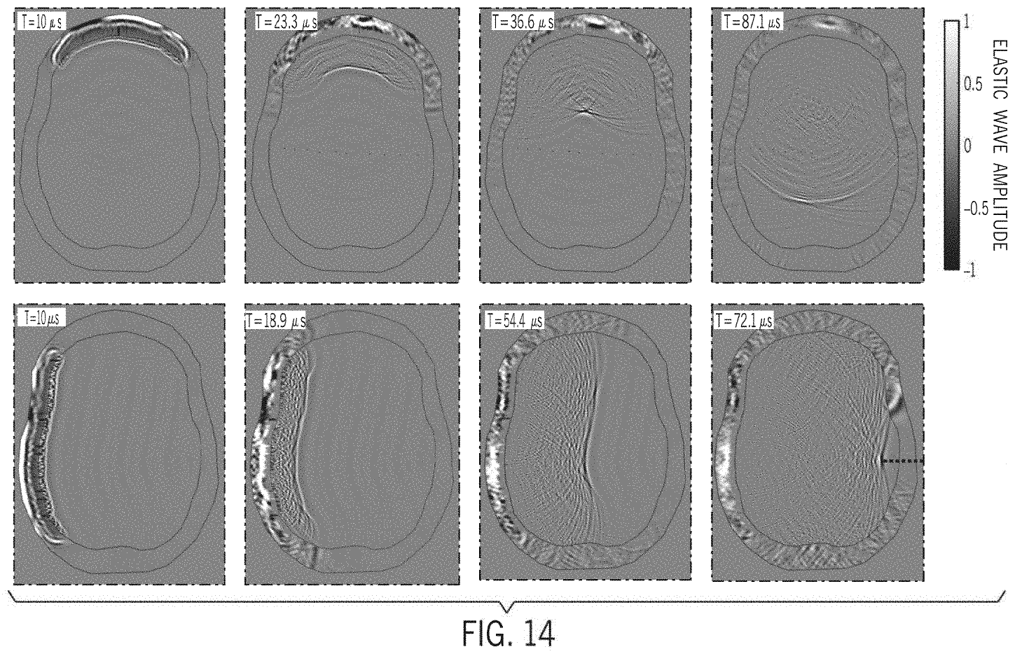

Referring to FIG. 14, non-limiting example ultrasonic wave propagation time snapshots from a 2D simulation are shown for frontal and side excitations of a subject's skull.

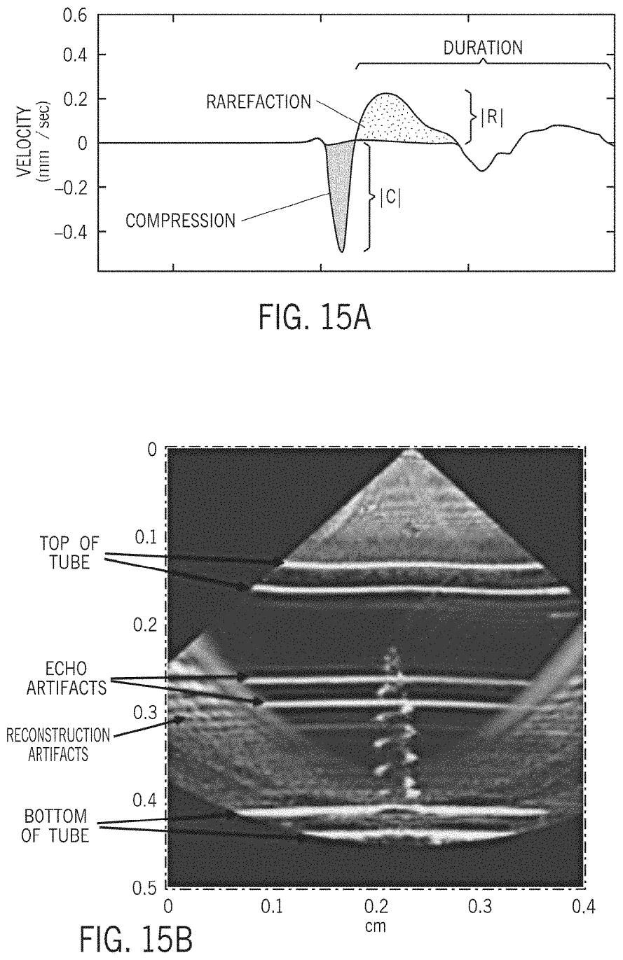

Referring to FIGS. 15A-15B, non-limiting example ultrasound signal products are shown. FIG. 15A shows non-image based, time-series signals using wavelet analysis. In a non-limiting example, the wavelet may be analyzed for wavelet duration, compression, rarefaction, polarity (If |R|>|C|, then polarity=1; if |R|<|C|, then polarity=−1), and the ratio of the wavelet area of rarefaction to the area of compression. These metrics may provide diagnostic information related to bleeding viscosity, pressure, and temperature.

FIG. 15B shows an example anatomical image using synthetic aperture US (SAUS). In this example, an ABS tube phantom with an internal drywall screw is depicted, wherein the image is acquired as immersed in water (10×averaging).

The present disclosure has described one or more preferred embodiments, and it should be appreciated that many equivalents, alternatives, variations, and modifications, aside from those expressly stated, are possible and within the scope of the invention.

1. A method for generating at least one of an image, or a tissue map of a subject, and/or providing diagnostic information characterizing interior tissue disease with the method comprising:
transmitting electromagnetic (EM) waves to a subject without patient contact, external to the human body;
generating thermoelastic acoustic propagating waves inside the subject using the radio frequency waves as the source;
detecting and measuring the acoustic propagating waves using an optical device or a contact transducer system to sense, temporally measure, and spatially map acoustic/mechanical vibrational waves; and
construction of at least one image, tissue characterization or report of the subject based on the sensed and measured acoustic propagating waves.

2. The method of claim 1, wherein the EM waves can be generated with a phased array or shaped horn antenna that in turn can steer and shape the acoustic radiation pattern of the acoustic waves in the soft tissue on the opposite side of the bone or high acoustic impedance barrier.

3. The method of claim 1, wherein the thermoelastic acoustic propagating waves include at least one of a longitudinal or compressional or ultrasound wave or shear wave.

4. The method of claim 1, wherein the optical sensing system includes at least one of a coherent laser vibrometer, light detection and ranging (LIDAR) detector, optical camera, a short wavelength infrared camera (SWIR), or a diffuse correlation spectroscopy (DCS) system.

5. The method of claim 4, wherein the optical detection system includes a wavelength in a range of 700-1064 nm.

6. The method of claim 1, wherein transmitting the EM waves from a single horn antenna or phased array of EM actuators includes applying pulsed EM energy, less than 10 microseconds in duration and at a pulse repetition frequency configured to be converted to the propagating waves after passing into the subject.

7. The method of claim 1, wherein the subject includes soft tissue, or a complex of bone and soft tissue and the propagating waves propagate in a tissue of the subject.

8. The method of claim 1, further comprising quantitative ultrasound techniques and elastography techniques to generate an image or report of the subject using the detected and measured propagating ultrasonic or audible band acoustic waves.

9. The method of claim 8, wherein the image or report of the subject using the detected and measured propagating audible band acoustic wave include synthetic aperture ultrasonic image construction.

10. The method of claim 8, wherein the image or report of the subject using the detected and measured propagating audible band acoustic wave includes acoustic wavelet analysis of single time series measurements.

11. The method of claim 1, wherein the EM waves are transmitted in a frequency range of 20 kHz-10 GHz.

12. The method of claim 1, further comprising determining at least one of a frequency of the EM waves or a wavelength accounting for at least one of: Specific Absorption Rate (SAR), Mechanical Index (MI), tissue heating, or optical safe parameters.

13. The method of claim 1, wherein the optical detection system is at least one of swept or ramped to provide for range binning of the detected propagating waves to determine a depth of a feature in the subject.

14. The method of claim 1, wherein the contact transducer system is attached to an exterior surface of the subject's scalp and measures the acoustic propagating waves on the exterior surface. 15 The method of claim 1, wherein the contact transducer system at least one of a wearable device and a flexible ultrasound receiver surface device.

16. A method for generating at least one of an image or a map of a subject, the method comprising:
delivering a first electromagnetic radiation to a first material in the subject;
converting the first electromagnetic radiation to an acoustic radiation force to transmit within a second material in the subject;
detecting transmission of the acoustic radiation force within the second material in the subject to acquire data; and
constructing an image or a map of the subject from the data.

17. The method of claim 16, wherein the first electromagnetic radiation includes an EM wave and the acoustic radiation includes one of an ultrasound or audible band acoustic wave—longitudinal or a shear wave.

18. The method of claim 16, wherein detecting includes using an optical sensor or a contact transducer.

19. The method of claim 18, wherein using the optical sensor includes using is at least one of a coherent laser vibrometer, light detection and ranging (LIDAR) detector, visible band camera, a short wavelength infrared camera (SWIR), or a diffuse correlation spectroscopy (DCS) system.

20. The method of claim 18, wherein using the optical sensor includes using a wavelength in a range of 700-1064 nm.

21. The method of claim 18, wherein the contact transducer is attached to an exterior surface of the subject's scalp and measures the acoustic radiation force on the exterior surface.

22. The method of claim 18, wherein the contact transducer is at least one of a wearable device and a flexible ultrasound receiver surface device.

23. The method of claim 16, wherein delivering the first electromagnetic radiation includes applying pulses of the first electromagnetic radiation configured to be converted to the acoustic radiation via thermoelastic mechanisms after passing through the first material.

24. The method of claim 16, wherein the first material is bone and the second material is tissue.

25. The method of claim 16, further comprising quantitative ultrasound techniques or elastography techniques generating an image or report of the subject using the acquired data.

26. The method of claim 16, wherein the first electromagnetic radiation includes radio frequency (RF) waves transmitted in a frequency range of 20 kHz -10 GHz.

27. The method of claim 16, further comprising determining at least one of a frequency of the first electromagnetic radiation or a wavelength for detecting transmission of the acoustic radiation force by accounting for at least one of: Specific Absorption Rate (SAR), Mechanical Index (MI), tissue heating, or optical safe parameters.

28. A system for constructing at least one of an image or a map of a subject, the system comprising:
a first electromagnetic radiation transmitter for delivering a first electromagnetic radiation to a first material in the subject;
wherein the first electromagnetic radiation is configured to convert to an acoustic radiation force to transmit within a second material in the subject;
a detector for detecting transmission of the acoustic radiation force within the second material in the subject to acquire data; and
a computer system configured to generate and construct an image or a map of the subject from the data.

29. The system of claim 28, wherein the first electromagnetic radiation includes an EM wave and the acoustic radiation includes one of an ultrasound or audible band acoustic wave including longitudinal waves or a shear wave.

30. The system of claim 28, wherein the detector includes an optical sensor or a contact transducer.

31. The system of claim 30, wherein the optical sensor includes at least one of a coherent laser vibrometer, light detection and ranging (LIDAR) detector, visible band camera, a short wavelength infrared camera (SWIR), or a diffuse correlation spectroscopy (DCS) system.

32. The system of claim 30, wherein the optical sensor includes a wavelength in a range of 700-1064 nm.

33. The method of claim 30, wherein the contact transducer is attached to an exterior surface of the subject's scalp and measures the acoustic radiation force on the exterior surface.

34. The method of claim 30, wherein the contact transducer is at least one of a wearable device and a flexible ultrasound receiver surface device.

35. The system of claim 28, wherein the first electromagnetic radiation transmitter is configured to apply pulses of the first electromagnetic radiation configured to be converted to the acoustic radiation after passing through the first material.

36. The system of claim 28, wherein the first material is bone and the second material is tissue.

37. The system of claim 28, wherein the computer system is further configured to employ quantitative ultrasound techniques or elastography techniques to generate and construct an image or report of the subject using the acquired data.

38. The method of claim 37, wherein the image or report of the subject using the detected propagating audible band acoustic wave include synthetic aperture ultrasonic image construction.

39. The method of claim 37, wherein the image or report of the subject using the detected propagating audible band acoustic wave includes acoustic wavelet analysis of single time series measurements

40. The system of claim 28, wherein the first electromagnetic radiation includes radio frequency (RF) waves transmitted in a frequency range of 20 kHz-10 GHz.

41. The system of claim 28, wherein the computer system is further configured to determine at least one of a frequency of the first electromagnetic radiation or a wavelength for detecting transmission of the acoustic radiation force by accounting for at least one of: Specific Absorption Rate (SAR), Mechanical Index (MI), tissue heating, or optical safe parameters.
